(19) United States
(12) Patent Application Publication
(10) Pub. No.: US 2004/0001800 A1
(43) Pub. Date: Jan. 1, 2004

(54) Hemo-and biocompatible polymeric adsorbing material, method of producing the material, and method of and device for purification of physiological fluids of organism with the use of the material

(76) Inventors: Vadlm Davankov, Moscow (RU); Ludmila Pavlova, Moscow (RU); Peter Quartararo, New York, NY (US); Maria Tsyurupa, Moscow (RU)

Correspondence Address: Ilya Zborovsky, 6Schoolhouse Way, Dix Hills, NY, 11746, US

(21) Appl. No.: 09/961,781

(22) Filed: Sep. 24, 2001

(51) Int. Cl. A61K 31/74; A61K 39/395; A61K 9/14; A61M 31/00

(52) U.S. Cl. 424/78.08; 424/140.1; 604/500

(57) ABSTRACT

A hemo- and biocompatible polymeric adsorbing material for purification of physiological fluids of organism has a plurality of beads each having a generally hydrophobic core provided with a plurality of pores, and a hydrophilic hemo- and biocompatible coating which coats at least a part of a surface of the core, wherein a thickness of the hemo- and biocompatible coating is selected between about 20 angstrom and about 1 micron. A method of producing the material includes the new coating of the surface of the core. A device for and a method of purification of physiological fluids of organism includes the use of the new material.

### BACKGROUND OF THE INVENTION

The present invention relates to a hemo- and biocompatible polymeric adsorbing material, a method of producing the material, and a method of and device for purification of physiological fluids of organism using of the material.

Porous hydrophobic natural and polymeric materials, in particular, activated carbon and macroporous polystyrene resins, are widely used in numerous adsorption technologies. They represent a good choice for purifying blood or other physiological fluids from many endogenic and exogenic toxic organic compounds. However, the hydrophobic nature of these materials can cause activation of multiple components of blood including the compliment and clotting systems and platelets. Therefore, in hemoperfusion procedures, only surface modified particles of the adsorbents can be employed. Modification means the formation of a surface layer or coating. However, the formation of the coating on the surface of the particles creates significant obstacles for diffusion of molecules to be adsorbed.

Materials which have a hydrophobic interior or core and hydrophilic and biocompatible coating or shell are disclosed, for example, in U.S. Pat. Nos. 4,410,652; 4,202,775; 5,773,384; 5,904,663; 6,087,300; 6,114,466; and 6,127,311. The application of the coating on the surface of the core of the beads of the material is performed by various methods which involve the formation of a hydrophilic hemo- and biocompatible shell and its retention on the surface of the core. Despite what appears to be the highly beneficial results described in the prior art, it is believed that the they can be further improved.

### SUMMARY OF THE INVENTION

Accordingly, it is an object of present invention to provide an improved hemo- and biocompatible polymeric adsorbing material, a method of producing the material, and a method of and device for purification of physiological fluids of organism with the use of the material.

In keeping with these objects and with others which will become apparent hereinafter, one feature of present invention resides, briefly stated, in a hemo- and biocompatible polymeric adsorbing material for purification of physiological fluids of organism which has a plurality of beads each having a generally hydrophobic core provided with a plurality of pores, and a hydrophilic hemo- and biocompatible coating which coats at least a part of a surface of the core, wherein a thickness of the coating is between about 20 angstrom and 1 about micron.

In accordance with another feature of present invention, a method of producing a beaded hemo- and biocompatible polymeric adsorbing material for purification of physiological fluids of organism is proposed, which includes the steps of producing a generally hydrophobic core of the beads provided with a plurality of pores, and applying a hydrophilic hemo- and biocompatible coating which coats at least a major part of a surface of the core, wherein a thickness of the coating is between about 20 angstrom and about 1 micron.

Still a further feature of the present invention is a device for purification of physiological fluids of organism which includes a container having an inlet for introducing a physiological fluid into the container to be purified, an outlet for withdrawing the purified fluid from the container, and in the container a body of hemo- and biocompatible polymeric adsorbing material comprising a plurality of beads each having a generally hydrophobic core provided with a plurality of pores, and a hydrophilic hemo- and biocompatible coating which covers at least a part of a surface of the core, wherein a thickness of the coating is between about 20 angstrom and about 1 micron.

It is another feature of the present invention to provide a method of purification of physiological fluids of organism, which includes the steps of passing a physiological fluid through a biocompatible polymeric adsorbing material which includes a plurality of beads each having a generally hydrophobic core provided with a plurality of pores, and a hydrophilic hemo- and biocompatible coating which covers at least a part of a surface of the core, wherein a thickness of the coating is between about 20 angstrom and about 1 micron which provides for efficient diffusion of molecules of toxins having a predetermined size through the coating at a predetermined speed of passage of the physiological fluid through the material.

When the physiological fluid of organism is passed through the inventive material, molecules of toxins contained in the physiological fluid of organism efficiently diffuse through the hemo- and biocompatible coating and are adsorbed by the core of the beads.

With the thickness of the hemo- and biocompatible coating between about 20 angstrom and about 1 micron, toxins within a broad range of molecular weights, for example low molecular weight toxins with a molecular weight about 100-500 Da and toxins with middle molecular weight within the range about 500-40,000 Da readily diffuse through the coating of the beads when the blood passes through the material with a speed of about 200-500 ml/min.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view showing a dependence of Cytochrom C adsorption on the thickness of the coating;

FIG. 2 is a view showing a platelet response during hemoperfusion in vivo.

### DESCRIPTION OF PREFERRED EMBODIMENTS

In accordance with the present invention, a hemo- and biocompatible polymeric adsorbing material is proposed, which has a plurality of beads each having a generally hydrophobic core provided with a plurality of pores, and a hydrophilic hemo- and biocompatible coating which coats at least a part of a surface of the core, wherein a thickness of the coating is between about 20 angstrom and about 1 micron.

A physiological fluid of organism such as blood has small molecular weight toxins within the range about 100-500 Da and middle molecular weight toxins within the range about 500-40,000 Da. For passage through the coating of small molecular weight toxins a thicker coating is suitable, while for passage through the coating of the middle molecular weight toxins a thinner coating is needed since diffusion of middle size molecules proceeds slower with the same speed of passage of the blood through the material.

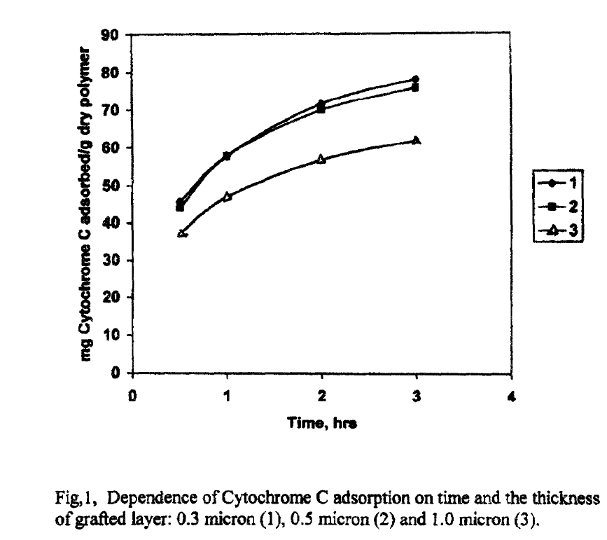

This is shown in FIG. 1 which illustrates the dependence of cytochrome C adsorption over time of coating with the thickness of 20 angstrom, 0.3 micron, 0.5 micron and 1 micron under the following conditions: 37° C., 250 mg wet sample, 15 ml of 0.5 mg/ml cytochrom C in 0.05 M tris-buffer, pH=7.4 and beads with a diameter of 500-600 micron.

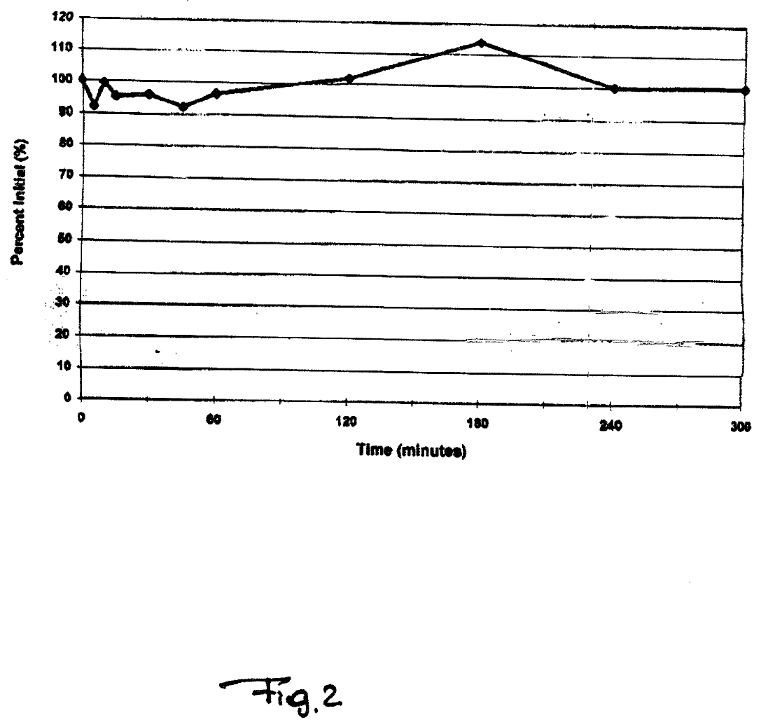

FIG. 2 shows the result of a canine test which was conducted for 5 hours. The blood was passed through a cartridge filled with the novel material in which the core of the beads was coated with the coating of 20 angstrom. The test results demonstrate that during the test the quantity of platelets was reduced by a very small amount at the beginning of the test and then increased, and returned to the pretest value by the end of the test.

TABLE 1
(presents the data obtained from FIG. 1)

| Time | Adsorption of Cytochrome C onto coated beads | | | |
|---|---|---|---|---|
| hours | 20 angstrom | 0.3 micron | 0.5 micron | 1 micron |
| 0.5 | 51.3 | 45.2 | 43.5 | 36.9 |
| | 50.9 | 46.2 | 44.5 | 37.4 |
| 1 | 63.0 | 57.9 | 57.8 | 46.7 |
| | 63.5 | 57.6 | 57.5 | 47.1 |
| 2 | 79.2 | 71.4 | 68.1 | 56.5 |
| | 79.8 | 71.4 | 69.9 | 56.6 |
| 3 | 88.7 | 77.7 | 75.5 | 61.6 |
| | 89.2 | | | |

Adsorption values are given in mg of cytochrom C per 1 g of dry polymer.

In order to produce the beads having a hydrophobic core with the hemo- and biocompatible coating having a desired thickness, it is necessary to determine the quantity of the initial material of the coating. This can be done for example as described below.

Total outer surface of 100 g beads having a diameter of 0.5 mm (0.05 cm) was calculated as follows:

The outer surface of 1 bead: S₁=4πR²=4·3.14·0.025²=0.00785 cm². The volume of 1 bead V₁=4/3πR³=4/3·3.14·0.025³=0.000065 cm³. The density of polymer is 1 g/cm³. Thus 100 g of beads have the volume of 100 cm³. The number of beads in 100 g (100 cm³):

$$\frac{100}{6.5 \cdot {10}^{- 5}} = 1.5 \cdot {10}^{6}$$

The outer surface of 100 g beads is 1.5·10⁶·0.00785=12,000 cm².

1.2 ml of monomer (or comonomers) of the coating grafted to the outer surface of 100 g beads, forms a layer with a thickness:

$$\frac{1.2}{12 , 000} = 1 \cdot {10}^{- 4} cm = 1 μm$$

The smaller amount of grafted monomers (or comonomers) produces a layer with a smaller thickness.

The hemo- and biocompatible coating on the core of the beads in accordance with the present invention can be formed as a monolayer for the coating thickness, for example, from about 20 angstrom to about 1000 angstrom, or as polylayers for the coating thickness, for example, from about 1000 angstrom to about 1 micron. Even within the second or thicker range it is possible to create a monolayer if the coating is composed of a high molecular weight polymeric material. Also, if the coating is close to 1 micron, linear macromolecules can be used, and when the coating is thinner than 1 micron, linear or crosslinked macromolecules can be used.

The thickness of the hemo- and biocompatible coating can be chosen to suit specific applications. For example, in emergency situations such as sepsis syndrome, when it is necessary to purify blood to avoid a fatal outcome, the speed of toxin removal must be maximal. Therefore it is recommended to use the material with the beads having a hemo- and biocompatible coating with a thickness in the range of about 20-100 angstrom. When the patients have a high level of toxins, such as beta-2 microglobulin in the blood, but they are not in a critical condition, it is recommended to use the material with the beads having a hemo- and biocompatible coating with a thickness in the range of about 100 angstrom to about 0.5 micron. For chronic patients who are in a weakened condition and the highest degree of hemo- and biocompatibility is important, it is recommended to use the material with the beads having a hemo- and biocompatible coating with a thickness in the range of about 0.5-1 micron.

The material in accordance with the present invention is produced by the inventive method as illustrated by the following examples.

### EXAMPLE 1

Into a seven-liter four-necked round-bottom flask equipped with a stirrer, a thermometer and a reflux condenser, there was placed the solution of 8.4 g polyvinyl alcohol-type technical grade emulsion stabilizer GM-14 in four liters of deionized water (aqueous phase). A solution of 260 ml divinylbenzene, 140 ml ethylvinylbenzene, with 250 ml toluene and 250 ml n-octane as porogens, and 2.94 g benzoyl peroxide (organic phase) was then added to the aqueous phase while stirring at room temperature. In 20 min, the temperature is raised to 80° C. The reaction was carried out at 80° C. for 8 hours and 90-92° C. for an additional 2 hours. After completing the copolymerization, the stabilizer was rigorously washed out with hot water (60 to 80° C.). The liquid was removed from the reactor and a solution of 5g trisodium phosphate in 3 L water was added. The temperature was raised to 80°, a solution of 10.2 g of ammonium persulfate in 40 ml water was added and in a few minutes a solution of 1.8 ml of vinyl pyrrolidone in 100 ml H₂O was introduced. The reaction took place over 3 hours at 70° C. while stirring. After completing the reaction, the polymer was washed with water and the organic solvents were removed by steam distillation. The beads obtained were filtered, and washed with 1 L of dioxane and with deionized water. Finally, the beads were dried in an oven at 60° C. overnight.

The polymer obtained in Example 1

1. displayed an inner surface area of 1200 sq.m/g and a total pore volume of 0.8 ml/g;

2. increased its volume in ethanol by a factor of 1.3;

3. efficiently removed beta-2-microglobulin from the blood of patients on chronic dialysis treatment;

4. successfully passed the hemocompatibility test (recalcification of plasma within the allowed 126-144 sec time limit);

5. individual spherical beads of the polymer with a diameter of 0.4-0.63 mm were mechanically destroyed at a loading of 450±50 g. This result is an improvement ver typical macroporous beads which were destroyed at a loading of about 120-150 g, but not as good as typical hypercrosslinked beads (up to 600 g) of a comparable diameter and total pores volume.

The thickness of the coating was 0.42 micron.

### EXAMPLE 2

Following the procedure of Example 1, 4.32 ml of N-vinyl-2-pyrrolidone was utilized for coating. The inner surface area of the product obtained was 1000 sq.m/g. The volume swelling with ethanol 1.25. The thickness of the coating was 1 micron.

### EXAMPLE 3

Following the procedure of Example 1, 2.2 ml N-vinyl-2-pyrrolidone was employed for the coating. Inner surface area of the product obtained was 1150 sq.m/g. Volume swelling with ethanol was 1.5. The thickness of the coating was 0.5 micron.

### EXAMPLE 4

Following the procedure of in Example 1, 2.0 ml N-vinyl-2-pyrrolidone in aqueous phase was utilized for the coating and an organic phase was utilized consisting of 320 ml divinylbenzene, 80 ml ethylvinylbenzene, 600 ml toluene and 600 ml n-octane as porogens, and 2.94 g bis-azoisobuthyro nitrile. N-vinyl-2-pyrrolidone grafting was about 4% of the initial quantity. The inner surface area of the product obtained was 800 sq.m/g. The volume swelling with ethanol was 1.3. The thickness is 0.016 micron. It is a monomolecular layer of polymer with a molecular weight of 60,000- 65,000.

### EXAMPLE 5

To a 14 L reactor 7.7 L of H₂O, and 12.2 g of stabilizer were added. When the stabilizer had dissolved at 70° C., 14.6 g monosodium phosphate, 48.8 g of disodium phosphate, 30.0 g trisodium phosphate, 75 g NaCl and 135 mg of NaNO₂ were added. Then a solution of 1500 ml 63% DVB, 10.4 g benzoyl peroxide, 1050 ml of toluene and 1500 ml of iso-octane were added to the reactor with stirring. The reaction was carried out at 80° C. for 12 hours. Then the stabilizer was washed out with hot-water and afterwards the beads were washed with isopropanol and water. The latter was removed and a new portion of 1800 ml of water, was added to the reactor. A solution of 40.6 g ammonium persulfate in 140 ml H₂O, the solution of 35 ml of TEMED in 500 ml water and 2.2 ml of N-vinyl-2-pyrrolidone in 650 ml H₂O were added successively. The reaction was carried out at 40° C. for 2 hours. The beads were washed with water. N-vinyl-2-pyrrolidone grafting was 1.5%. The thickness of coating was about 20Å.

### EXAMPLE 6

To a 2 L reactor 1 L of H₂O, 10 g of Povidone having a molecular weight of 360 Kda, 6.3 g of disodium phosphate, 3.9 g of trisodiumphosphate and 30 mg of sodium nitrite were added. Then, a solution of 160 ml 63% DVB, 1.1 g of benzoyl peroxide, 110 ml of toluene and 160 ml of isooctane were poured into the reactor with stirring. The reaction was carried out at 800 for 11 hours. Afterwards 4.6 g of ammonium persulphate were added to the reaction mixture, 1 ml of N-vinyl-2-pyrrolidone in 20 ml of water was added and the reaction was continued for 3 hours at 70° C. The beads obtained were washed with water, isopropanol and water. The thickness of coating is about 1 micron.

The present invention also provides a device for purification of physiological fluids of organism which includes a container having an inlet for introducing a physiological fluid into the container to be purified, an outlet for withdrawing the purified fluid from the container, and in the container a body of biocompatible polymeric adsorbing material comprising a plurality of beads each having a generally hydrophobic core provided with a plurality of pores, and a hydrophilic hemo-and biocompatible coating which coats at least a part of the surface of the core, wherein a thickness of the coating is selected about 20 angstrom and about 1 micron.

Finally, the present invention provides a method of purification of physiological fluids of organism which includes the steps of passing a physiological fluid through a biocompatible polymeric adsorbing material which includes a plurality of beads each having a generally hydrophobic core provided with a plurality of pores, and a hydrophilic hemo- and biocompatible coating which coats at least a part of the surface of the core, wherein a thickness of the coating is between about 20 angstrom and about 1 micron.

It will be understood that each of the elements described above, or two or more together, may also find a useful application in other types of constructions differing from the types described above.

While the invention has been illustrated and described as embodied in hemo- and biocompatible polymeric adsorbing material, method of producing the material, and method of and device for purification of physiological fluids of organism with the use of the material, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A hemo- and biocompatible polymeric adsorbing material for purification of physiological fluids of organism, comprising a plurality of beads each having a generally hydrophobic core provided with a plurality of pores, and a hydrophilic hemo- and biocompatible coating which coats at least a part of a surface of the core, wherein a thickness of the hemo- and biocompatible coating is between about 20 angstrom and about 1 micron.

2. A hemo- and biocompatible polymeric adsorbing material as defined in claim 1, wherein the hemo- and biocompatible coating is composed of a monomolecular layer of a coating material.

3. A hemo- and biocompatible polymeric adsorbing material as defined in claim 1, wherein the hemo- and biocompatible coating is composed of a polymolecular layer of a coating material.

4. A hemo- and biocompatible polymeric adsorbing material as defined in claim 1, wherein the hemo- and biocompatible coating has the thickness of between about 20 angstrom and about 100 angstrom.

5. A hemo- and biocompatible polymeric adsorbing material as defined in claim 1, wherein the hemo- and biocompatible coating has the thickness of between about 100 angstrom and about 0.5 micron.

6. A hemo- and biocompatible polymeric adsorbing material as defined in claim 1, wherein the hemo- and biocompatible coating has the thickness of between about 0.5 micron and about 1 micron.

7. A method of producing a beaded hemo- and biocompatible polymeric adsorbing material for purification of physiological fluids of organism, comprising the steps of producing a core of the beads having hydrophobic property and provided with a plurality of pores; applying a hydrophilic hemo- and biocompatible coating which coats at least a part of a surface of the core, and a thickness of the hemo- and biocompatible coating is between about 20 angstrom and about 1 micron.

8. A method as defined in claim 7, wherein said applying includes applying a monomolecular layer of a coating material.

9. A method as defined in claim 7, wherein said applying includes applying a polymolecular layer of a coating material.

10. A method as defined in claim 7, wherein said applying includes applying the hemo- and biocompatible coating with a thickness of between about 20 and about 100 angstrom.

11. A method as defined in claim 7, wherein said applying includes applying the hemo- and biocompatible coating with a thickness of between about 100 angstrom and about 0.5 micron.

12. A method as defined in claim 7, wherein said applying includes applying the hemo- and biocompatible coating with a thickness of between about 0.5 micron and about 1 micron.

13. A device for purification of physiological fluids of organism, comprising a container having an inlet for introducing a physiological fluid to be purified into the container, and an outlet for withdrawing the purified fluid from the container, and in the container a body of hemo- and biocompatible polymeric adsorbing material comprising a plurality of beads each having a generally hydrophobic core provided with a plurality of pores, and a hydrophilic hemo- and biocompatible coating which coats at least a part of a surface of the core, wherein a thickness of the coating is between about 20 angstrom and about 1 micron.

14. A method of purification of physiological fluids of organism, comprising passing a physiological fluid through a body of hemo-and biocompatible polymeric adsorbing material which includes a plurality of beads each having a generally hydrophobic core provided with a plurality of pores, and a hydrophilic hemo- and biocompatible coating which coats at least a part of a surface of the core, wherein a thickness of the coating is between about 20 angstrom and about 1 micron.
